(19) United States
(12) Patent Application Publication
(10) Pub. No.: US 2023/0390270 A1
(43) Pub. Date: Dec. 7, 2023

(54) METHODS FOR TREATING REMITTING MULTIPLE SCLEROSIS

(71) Applicant: BIOGEN MA INC., Cambridge, MA (US)

(72) Inventors: Shibeshih Belachew, Cambridge, MA (US); Ellen Cahir-McFarland, Cambridge, MA (US); Zhaohui Shao, Cambridge, MA (US); Hui-Hsin Tsai, Cambridge, MA (US); Ru Wei, Cambridge, MA (US)

(21) Appl. No.: 18/021,643

(22) PCT Filed: Aug. 19, 2021
(86) PCT No.: PCT/US2021/046589

§ 371 Date: Feb. 16, 2023

Related U.S. Application Data
(60) Provisional application No. 63/067,727, filed on Aug. 19, 2020.

(51) Int. Cl. A61K 31/445; A61K 45/06; A61P 9/10

(52) U.S. Cl. CPC A61K 31/445; A61P 9/10; A61K 45/06

(57) ABSTRACT

Disclosed is a method of treating a human subject with multiple sclerosis. The method comprises administering to the subject in the absence of a cholesterol lowering drug an effective amount of Compound 1: or a pharmaceutically acceptable salt thereof.

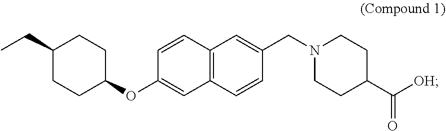 [SMILES: CC[C@H]1CC[C@@H](Oc2ccc3cc(CN4CCC(C(=O)O)CC4)ccc3c2)CC1]

### RELATED APPLICATION

This application claims the benefit of the filing date, under 35 U.S.C. § 119(e), of U.S. Provisional Application No. 63/067,727, filed on Aug. 19, 2020, the entire contents of which are incorporated here by reference.

### BACKGROUND

Multiple sclerosis (MS) is a chronic, disabling neurological disease that affects an estimated 1 million people in North America and Western Europe. It is characterized by prominent demyelination and axonal loss affecting the white and gray matter of the brain, spinal cord, and the optic nerve and manifests clinically by loss of neurological function that can occur in a relapsing-remitting pattern and/or as sustained progression over time. The majority of patients with untreated MS develop irreversible disability, including the loss of independent ambulation within 15 years of diagnosis.

Remyelination is a critical natural process in the human CNS. It has been known for more than 30 years that, in MS, prolific and widespread remyelination takes place in both acute and chronic active lesions. Prineas J W, Connell F. Ann Neurol. 1979; 5(1):22-31. This process of remyelination occurs by differentiation of oligodendrocyte progenitor cells (hereinafter “OPCs”) Blakemore W F, Keirstead H S, J Neuroimmunol. 1999; 98(1):69-76; Chang A, Nishiyama A, Peterson J, et al. J Neurosci. 2000; 20(17):6404-12; Dawson M R, Levine J M, Reynolds R. J Neurosci Res. 2000; 61(5):471-9; Lucchinetti C, Bruck W, Parisi J, et al. Brain 1999; 122 (Pt 12):2279-95; Raine C S, Moore G R, Hintzen R, et al. Lab Invest. 1988; 59(4):467-76; and Scolding N, Franklin R, Stevens S, et al. Brain 1998; 121 (Pt 12):2221-8.

Histologically, remyelination results in the formation of “shadow plaques” that corresponds to areas of a prior demyelination with new formation of disproportionally thin myelin sheaths with short internodes. Remyelination has been shown to restore conduction block in experimental animals. Smith K J, Blakemore W F, McDonald W I, Brain 1981; 104(2):383-404. Restoration of conduction block is likely responsible for the remitting aspect that characterizes most MS attacks early in the course of the relapsing-remitting form of the disease. However, remyelination fails over time in the majority of patients with MS, as indicated by the finding of little, if any, remyelination in most chronic MS lesions examined at autopsy. Chang A, Tourtellotte W W, Rudick R, et al., N Engl J Med. 2002; 346(3):165-73; Chang K H, Lyu R K, Chen C M, et al., Mult Scler. 2006; 12(4):501-6.

Several immunomodulatory medications are approved for treatment of MS, including 5 interferon (IFN)-β therapies (3 IFN beta-1α and 2 IFN beta-1β therapies), glatiramer acetate, natalizumab, mitoxanthrone, fingolimod, teriflunomide, dimethyl fumarate, alemtuzumab, cladribine, ocrelizumab, siponimod, ozanimod, and diroximel fumarate. Although all of these disease modifying therapies are anti-inflammatory therapies that can reduce the frequency and severity of new MS lesions that could cause clinical exacerbations and disability, none of them is known to enhance the natural repair in damaged MS tissues. Therefore, there is a high unmet need for MS therapies that specifically improve tissue repair, notably remyelination and preservation of axons and their neurons.

### SUMMARY

It has now been found that Compound 1, whose structure is shown below, is an inhibitor of multiple enzymes in the cholesterol biosynthesis pathway, including LBR/TM7SF2 and EBP. Specifically, Compound 1 reduces cholesterol levels in healthy human volunteers in a dose dependent manner (Example 1) and causes accumulation of 7-dehydrocholesterol (7-DHC). The accumulation of 7-DHC is also replicated in rat OPCs treated with Compound 1 (Example 2). Compound 1 enhanced remyelination in rat lysophosphatidyl choline induced spinal cord and corpus callosum demyelination models and a mouse cuprizone demyelination model (see Example 3). Compound 1 also leads to robust myelination in an OPC/rat dorsal root ganglion co culture assay in a dose dependent manner (see Example 4) and enhanced differentiation of human iPSC-derived OPC to myelinating oligodendrocytes (see Example 5).

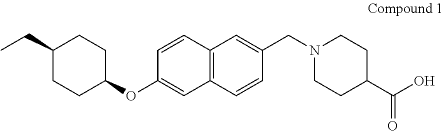 [SMILES: CC[C@H]1CC[C@@H](Oc2ccc3cc(CN4CCC(C(=O)O)CC4)ccc3c2)CC1]

### 1-((6-(((1s,4s)-4-ethylcyclohexyl)oxy)naphthalen-2-yl)methyl)piperidine-4-carboxylic acid

Based on these discoveries, methods of treating multiple sclerosis are disclosed herein; because of the cholesterol lowering effects of Compound 1, the methods are carried out in the absence of another cholesterol lowering drug. Alternatively, if a cholesterol lowering drug is co-administered with Compound 1, plasma cholesterol levels in the subject are monitored and the dose of the cholesterol lowering drug is adjusted, if necessary, to bring plasma cholesterol levels within a (desirable) target range.

Compound 1 is a known inhibitor of sphingosine-1-phosphate receptor 4 (hereinafter “S1P4”) and can be used for treating MS due to its re-myelination effects. See, for example, U.S. Pat. No. 9,340,527. While it was believed that Compound 1 effects on the S1P4 receptor could contribute to re-myelination, it has now been found that S1P4 has little if any expression in human CNS (including in human OPCs) derived from MS tissue, in contrast with rats (Example 6). From this result, it is unlikely that the remyelination effects of Compound 1 are mediated mainly through S1P4 in the CNS. Instead, as discussed above, it is now believed that the remyelinating effects of Compound 1 are at least in part due to its ability to engage the cholesterol biosynthesis pathway. Based on these discoveries, it is expected that Compound 1 can achieve OPC differentiation and re-myelination in humans at low doses, e.g., from 10 mg to 60 mg per day. It has been found from human trials that the risk of neutropenia is greater at higher doses outside of this range.

One embodiment of the disclosure is a method of treating a human subject with MS. The method comprises administering to the subject an effective amount of Compound 1 or a pharmaceutically acceptable salt thereof, in the absence of a cholesterol lowering drug.

Another embodiment of the disclosure is a method of treating a human subject with MS. The method comprises administering to the subject from 10 mg to 60 mg per day of Compound 1 (e.g., 10 mg to 20 mg per day, 20 mg to 30 mg per day, 30 mg to 40 mg per day, 40 mg to 50 mg per day or 50 mg to 60 mg per day) or an amount of a pharmaceutically acceptable salt thereof equivalent to 10 mg to 60 mg per day (e.g., 10 mg to 20 mg per day, 20 mg to 30 mg per day, 30 mg to 40 mg per day, 40 mg to 50 mg per day or 50 mg to 60 mg per day) of Compound 1.

Another embodiment of the disclosure is a method of treating a human subject with multiple sclerosis (MS), wherein the subject is being treated with an effective amount of a cholesterol lowering drug. The method comprises the steps of:
i) administering to the subject an effective amount of Compound 1:

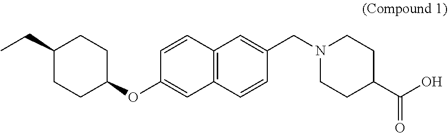 [SMILES: CC[C@H]1CC[C@@H](Oc2ccc3cc(CN4CCC(C(=O)O)CC4)ccc3c2)CC1]

or a pharmaceutically acceptable salt thereof;
ii) assessing the subject's plasma cholesterol level;
iii) if the subject's plasma cholesterol level is outside of a target range, adjusting the amount of cholesterol lowering drug administered to the subject to bring the subject's plasma cholesterol level within the target range. Steps ii) and iii) can be repeated until the subject plasma cholesterol level is within the target range.

Another embodiment of the disclosure is Compound 1 or a pharmaceutically acceptable salt thereof for treating a human subject with MS in the absence of a cholesterol lowering drug.

Another embodiment of the disclosure is Compound 1 or a pharmaceutically acceptable salt thereof for treating a human subject with MS in a subject with between 10 mg and 60 mg per day (e.g., between 10 mg and 20 mg per day, between 20 mg and 30 mg per day, between 30 mg and 40 mg per day, between 40 mg and 50 mg per day or between 50 mg and 60 mg per day) of Compound 1 or an amount of a pharmaceutically acceptable salt thereof equivalent to 10 mg to 60 mg per day (e.g., 10 mg to 20 mg per day, 20 mg to 30 mg per day, 30 mg to 40 mg per day, 40 mg to 50 mg per day or 50 mg to 60 mg per day) of Compound 1.

Another embodiment of the disclosure is the use Compound 1 or a pharmaceutically acceptable salt thereof for the manufacture of a medicament for treating a subject with MS in the absence of a cholesterol lowering drug.

Another embodiment of the disclosure is the use of Compound 1 for the manufacture of a medicament for treating a human subject with MS wherein the subject is treated with from 10 mg to 60 mg per day of Compound 1 or an amount of a pharmaceutically acceptable salt thereof equivalent to 10 mg to 60 mg per day of Compound 1.

### BRIEF DESCRIPTION OF DRAWINGS

FIG. 1 shows a time course of reduction in circulating mean total cholesterol levels in healthy volunteers administered placebo, 10 mg, 30 mg or 60 mg per day of Compound 1 over a 28 day period.

FIG. 2 is a bar graph showing predicted steady-state concentrations of circulating cholesterol levels during treatment with Compound 1 for subject administered placebo, 1 mg, 3 mg, 10 mg, 30 mg or 60 mg per day of Compound 1 at pharmacodynamic steady-state. The predicted concentrations are from simulations based on data from three phase I trials of Compound 1.

FIG. 3 are bar graphs showing changes in the levels of 7-DHC, cholesterol and desmosterol in rat OPC cultures treated with Compound 1.

FIG. 4 is a diagram showing the biosynthetic pathway of cholesterol and desmosterol.

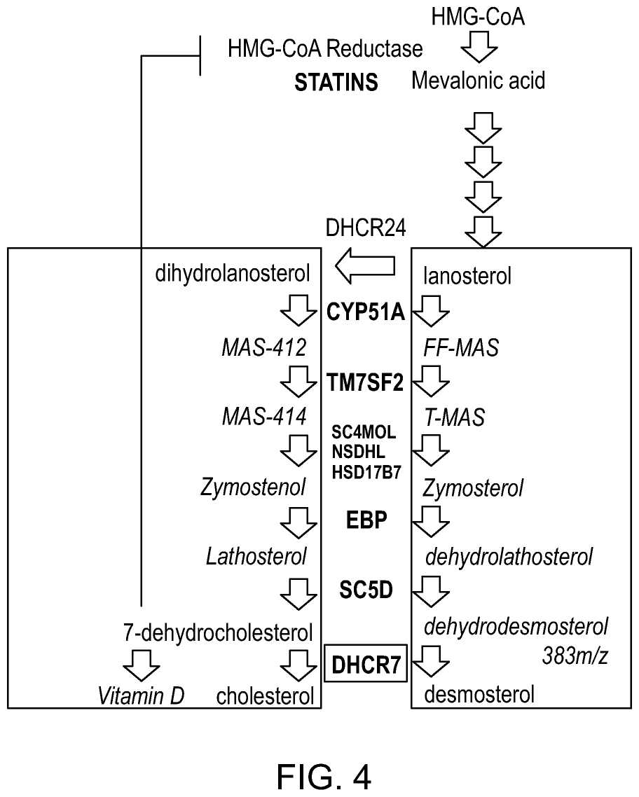

FIG. 5 is a bar graph showing that Compound 1 enhanced remyelination in the rat LPC spinal cord demyelination model in a dose-dependent manner.

FIG. 6 is a bar graph showing that Compound 1 enhanced dose-dependent remyelination in the mouse cuprizone model.

FIG. 7 is a photograph of an MBP Western blot used to determine myelination in OPC and dorsal root ganglion co-cultures.

FIG. 8A is a bar graph showing quantification of MBP+ cells in human iPSC-derived oligodendrocyte progenitor cells; p<0.0001 by unpaired t-test; and FIG. 8B is a bar graph showing quantification of MBP+ myelinated axon clusters in human iPSC-derived oligodendrocyte progenitor cells; p<0.01 by 1-way analysis of variance.

FIG. 9 is a bar graph showing S1P4 expression in various types of rat cells.

### DETAILED DESCRIPTION

Treatment with Compound 1 inhibits cholesterol biosynthesis and therefore results in decreased levels of cholesterol, in particular, peripheral cholesterol, within the patient. Compound 1 also stimulates and enhances the generation of new oligodendrocytes and intrinsic myelination and/or remyelination. As such, it is expected that Compound 1 is an effective treatment for MS. However, because of its inhibition of cholesterol biosynthesis, it is desirable to administer Compound 1 to MS patients “in the absence of a cholesterol lowering drug”. Alternatively, Compound 1 is co-administered with a cholesterol lowering drug with monitoring of the subject's plasma cholesterol levels to assess whether the subject's plasma cholesterol levels are within a target range determined to be desirable or normal for the subject. If the plasma cholesterol levels are outside the target range, the amount of cholesterol lowering drug being administered is adjusted to bring the plasma cholesterol levels within the target range. Cholesterol carries out various important biological functions in the body, including as an essential structural component of cell membranes and as a precursor for the biosynthesis of steroid hormones, bile acids, and vitamin D. In addition, cholesterol is an essential lipid component of myelin. Cholesterol does not cross the blood-brain-barrier, and the central nervous system is dependent on local de novo synthesis of cholesterol. Defects or disruptions in brain cholesterol metabolism has been implicated in various neurodegenerative diseases, such as Alzheimer's disease (AD), Huntington's disease (HD), Parkinson's disease (PD) and some cognitive deficits typical of the old age (Juan Zhang and Qiang Liu, Protein Cell, 6(4): 254-264 (2015)). Therefore, it is critical to maintain the cholesterol level of the patient within a target range when the patient is treated with Compound 1. In accordance with the methods described herein, in some embodiments, Compound 1 or a pharmaceutically acceptable salt thereof decreases cholesterol in the periphery in humans but does not significantly decrease cholesterol in the central nervous system based on animal data.

Administering “in the absence of a cholesterol lowering drug” means that the subject was never taking a cholesterol lowering drug or stopped taking a cholesterol lowering drug before treatment with Compound 1 or a pharmaceutically acceptable salt thereof was initiated or stopped taking a cholesterol lowering drug at the time treatment with Compound 1 or a pharmaceutically acceptable salt thereof was initiated. When the subject is taking a cholesterol lowering drug, it is desirable that administration of the cholesterol lowering drug be terminated at 1, 2, 3, 4, 5 or 6 days before initiation of treatment with Compound 1 or a pharmaceutically acceptable salt thereof; or at least 1, 2, 3, 4, 5, 6, 7, 8 or more weeks before initiation of treatment with Compound 1 or a pharmaceutically acceptable salt thereof; or at least 1, 2 or 3 months before initiation of treatment with Compound 1 or a pharmaceutically acceptable salt thereof.

A “cholesterol lowering drug” is a drug prescribed for and/or administered for the purpose of lowering cholesterol in human patients with elevated cholesterol levels. Examples include statins, PCSK9 inhibitors, selective cholesterol absorption inhibitors, bile acid sequestrants, fibrates or lipid-lowering therapies.

A statin is a cholesterol lowering drug that acts by inhibiting HMG-CoA reductase. Examples include atorvastatin (LIPITOR®), fluvastatin (LESCOL XL®), lovastatin (ALTOPREV®), pitavastatin (LIVALO®), pravastatin (PRAVACHOL®), rosuvastatin (CRSTOR®, EZALLOR™) and simvastatin (ZOCAR®, FLOLIPID®).

A PCSK9 inhibitor is a cholesterol lowering drug that acts by inhibiting proprotein convertase subtilisin/kexin type 9 serine protease. Examples include alirocumab and evolocumab.

A selective cholesterol absorption inhibitor is a cholesterol lowering drug that acts by inhibiting absorption of cholesterol in the intestines. For example, ezetimibe (ZEITA®) is a selective cholesterol abruption inhibitor that acts by acts by inhibiting the transporter, Niemann-Pick C-1-like 1 protein (NPC1L1).

A bile acid sequestrant is a cholesterol lowering drug that acts by binding bile acids in the intestine and increasing the excretion of bile acids in the stool. This reduces the amount of bile acids returning to the liver and forces the liver to produce more bile acids to replace the bile acids lost in the stool. In order to produce more bile acids, the liver converts more cholesterol into bile acids, which lowers the level of cholesterol in the blood. Examples include cholestyramine (QUESTRAN®, PREVALITE®), colestipol (COLESTID®) and colesevelam (WELCHOL®).

Fibric acid derivatives (fibrates) are a class of medication that lowers blood triglyceride levels. Fibrates lower blood triglyceride levels by reducing the liver's production of VLDL (the triglyceride-carrying particle that circulates in the blood) and by speeding up the removal of triglycerides from the blood. Fibrates also are modestly effective in increasing blood HDL cholesterol levels. Examples of fibrates includegemfibrozil (LOPID®) and fenofibrate (TRJICOR®, FIBRICOR®).

Other cholesterol lowering drugs include fish oils, niacin (nitotinic acid) cholestin, bempedoic acid (NEXLETOL®) and probucol.

A “target range” for a subject's plasma cholesterol level is a range determined to be desirable or normal for the subject or a range which is determined to be optimal for the subject's overall health and well-being. The target range is determined according to best practices among physicians treating hypercholesterolemia and can vary according to recommendations by medical professional organizations and government organizations, based on the most current research and experience in the field. A “target range” for a subject's plasma cholesterol can also vary, based on the subject's age and overall health. Generally, a normal range is from 100 mg/dL (milligrams per deciliter) to 200 mg/dL, but for certain subjects can be from 50 mg/dL to 200 mg/dL, 60 mg/dL to 200 mg/dL, 70 mg/dL to 200 mg/dL, 80 mg/dL to 200 mg/dL, 90 mg/dL to 200 mg/dL, 100 mg/dL to 200 mg/dL, 110 mg/dL to 200 mg/dL, 120 mg/dL to 200 mg/dL, 125 mg/dL to 200 mg/dL, 50 mg/dL to 175 mg/dL, 60 mg/dL to 175 mg/dL, 70 mg/dL to 175 mg/dL, 80 mg/dL to 175 mg/dL, 90 mg/dL to 175 mg/dL, 100 mg/dL to 175 mg/dL, 110 mg/dL to 175 mg/dL, 120 mg/dL to 175 mg/dL or 125 mg/dL to 175 mg/dL. Plasma cholesterol levels are routinely assessed during doctor office visits.

When a subject's plasma cholesterol level is outside of the target range, e.g., below the target range, the plasma cholesterol level can be adjusted by decreasing the dose of cholesterol lowering drug being administered to the subject. Conversely, when the plasma cholesterol level is above the target range, the plasma cholesterol level can be adjusted by increasing the dose of cholesterol lowering drug being administered to the subject.

Effective amounts of other drugs for treating MS can be co-administered with Compound 1 in the disclosed methods. Included are Tysabri®, dimethyl fumarate (e.g. Tecfidera®), diroximel fumarate (Vumerity®), monomethyl fumarate (e.g., Bafiertam), an interferon (such as pegylated or non-pegylated interferons, preferably interferon β-1α or pegylated interferon β-1α), glatiramer acetate, a compound improving vascular function, an immunomodulating agent (such as Fingolimod, cyclosporins, rapamycins or ascomycins, or their immunosuppressive analogs, e.g. cyclosporine A, cyclosporine G, FK-506, ABT-281, ASM981, rapamycin, 40-O-(2-hydroxy)ethyl-rapamycin etc.); corticosteroids; cyclophosphamide; azathioprine; mitoxanthrone, methotrexate; leflunomide; mizoribine; mycophenolic add; mycophenolate mofetil; 15-deoxyspergualine; difucortolone valerate; difuprednate; Alclometasone dipropionate; amcinonide; amsacrine; asparaginase; azathioprine; basiliximab; beclometasone dipropionate; betamethasone; betamethasone dipropionate; betamethasone phosphate sodique; betamethasone valerate; budesonide; captopril; chlormethine chlorhydrate; clobetasol propionate; cortisone acetate; cortivazol; cyclophosphamide; cytarabine; daclizumab; dactinomycine; desonide; desoximetasone; dexamethasone; dexamethasone acetate; dexamethasone isonicotinate; dexamethasone metasulfobenzoate sodique; dexamethasonephosphate; dexamethasone tebutate; dichlorisone acetate; doxorubicinee chlorhydrate; epirubicine chlorhydrate; fuclorolone acetonide; fludrocortisone acetate; fludroxycortide; flumetasone pivalate; flunisolide; fluocinolone acetonide; fluocinonide; fluocortolone; fluocortolone hexanoate; fluocortolone pivalate; fluorometholone; fluprednidene acetate; fluticasone propionate; gemcitabine chlorhydrate; halcinonide; hydrocortisone; hydrocortisone acetate; hydrocortisone butyrate; hydrocortisone hemisuccinate; melphalan; meprednisone; mercaptopurine; methylprednisolone; methylprednisolone acetate; methylprednisolone hemisuccinate; misoprostol; muromonab-cd3; mycophenolate mofetil; paramethansone acetate; prednazoline, prednisolone; prednisolone acetate; prednisolone caproate; prednisolone metasulfobenzoate sodique; prednisolone phosphate sodique; prednisone; prednylidene; rifampicine; rifampicine sodique; tacrolimus; teriflunomide; thalidomide; thiotepa; tixocortol pivalate; triamcinolone; triamcinolone acetonide hemisuccinate; triamcinolone benetonide; triamcinolone diacetate; triamcinolone hexacetonide; immunosuppressive monoclonal antibodies, e.g., monoclonal antibodies to leukocyte receptors, e.g., MHC, CD2, CD3, CD4, CD7, CD20 (e.g., ublituximab, rituximab and ocrelizumab), CD25, CD28, B7, CD40, CD45, CD56 (e.g., daclizumab), or CD58 or their ligands; or other immunomodulatory compounds, e.g. CTLA41g, or other adhesion molecule inhibitors, e.g. mAbs or low molecular weight inhibitors including Selectin antagonists and VLA-4 antagonists (such as Tysabri®). In another embodiment, the drug being co-administered with Compound 1 is interferon beta-1a, interferon beta-1(3, glatiramer acetate, mitoxantrone, natalizumab, fingolimod, teriflunomide, dimethyl fumarate, diroximel fumarate, alemtuzumab, ocrelizumab, siponimod, cladribine, ozanimod and ocrelizumab. In one aspect, interferon beta-1β and glatiramer acetate are used. When co-administered with another drug effective for treating MS, Compound 1 and the other drug can be administered at the same time (in the same or different formulations) or at different times.

“Effective amount” means an amount of a drug that alleviates one or more symptoms of a disease or condition and/or slows the progression of the disease or condition. With respect to Compound 1 used to treat MS, an “effective amount” includes an amount that induces OPC differentiation and remyelination in a human subject with MS. Exemplary effective amounts for Compound 1 in MS include, but are not limited to, 10 mg to 60 mg per day (or an amount of a pharmaceutically acceptable salt of Compound 1 equivalent to 10 to 60 mg of Compound 1), e.g., 10 mg per day, 30 mg per day or 60 mg per day. Exemplary effective amounts of pharmaceutically acceptable salts of Compound 1 include, but are not limited to, an amount equivalent to 10 mg per day to 60 mg per day of Compound 1, e.g., an amount equivalent to 10 mg per day, 30 mg per day or 60 mg per day of Compound 1. In some embodiments, an effective amount for Compound 1 can be 10 mg to 20 mg per day, 20 mg to 30 mg per day, 30 mg to 40 mg per day, 40 mg to 50 mg per day or 50 mg to 60 mg per day. In some embodiments, an effective amount of a pharmaceutically acceptable salt of Compound 1 can be an amount equivalent to 10 mg to 20 mg per day, 20 mg to 30 mg per day, 30 mg to 40 mg per day, 40 mg to 50 mg per day or 50 mg to 60 mg per day of Compound 1.

As used herein, when a range of values is expressed, it includes both endpoints. For example, an amount of 10 mg to 60 mg includes 10 mg and 60 mg. Similarly, an amount between 10 mg and 20 mg includes 10 mg and 20 mg.

“Subject” and “patient” may be used interchangeably, and mean a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The disclosed methods can be used for all stages of MS, including relapsing multiple sclerosis (or relapsing form(s) of multiple sclerosis), relapsing-remitting multiple sclerosis, primary progress multiple sclerosis, secondary progressive multiple sclerosis and clinically isolated syndrome (hereinafter “CIS”).

Relapsing multiple sclerosis (or relapsing form(s) of multiple sclerosis) includes clinically isolated syndrome, relapsing-remitting multiple sclerosis and active secondary progressive multiple sclerosis.

Relapsing-remitting multiple sclerosis is a stage of MS characterized by unpredictable relapses followed by periods of months to years of relative quiet (remission) with no new signs of disease activity. Deficits that occur during attacks may either resolve or leave problems, the latter in about 40% of attacks and being more common the longer a person has had the disease. This describes the initial course of 80% of individuals with multiple sclerosis.

Secondary progressive multiple sclerosis occurs in around 65% of those with initial relapsing-remitting multiple sclerosis, who eventually have progressive neurologic decline between acute attacks without any definite periods of remission. Occasional relapses and minor remissions may appear. The most common length of time between disease onset and conversion from relapsing-remitting to secondary progressive multiple sclerosis is 19 years.

Primary progressive multiple sclerosis is characterized by the same symptoms of secondary progressive multiple sclerosis, i.e., progressive neurologic decline between acute attacks without any definite periods of remission, without the prior relapsing-remitting stage.

EDSS is an ordinal MS disability scale that captures changes in the neurological examination and long distance ambulation. Time 25 Foot Walk Test (T25FW) is a quantitative measure of short distance ambulatory capacity and is therefore sensitive in disabled patients to detect clinical progression based on walking ability. 9 Hole Peg Test (SHPT) is a quantitative measure of upper extremity function, which has been demonstrated to worsen in MS population across a wide range disability. In one embodiment, the disclosed methods provide for a statistically significant improvement over 12 weeks of treatment in one or more in the EDSS, T25FW or SHPT test in patient with an EDSS score of from 2.0 to 6.0 prior to treatment. In another embodiment, the disclosed methods provide for an at least 5%, 10%, 15% or 20% improvement over 12 weeks of treatment in one or more of the EDSS, T25FW or SHPT test in patients with an EDSS score of from 2.0 to 6.0 prior to treatment.

In another embodiment, the disclosed methods provide for a statistically significant change in normalized magnetization transfer ratio and diffusion tenor imaging radial diffusivity in total baseline nonenhancing T2 lesions over 42 weeks of treatment. In another embodiment, the disclosed methods provide for a statistically significant change in normalized T1 intensity and T1 hypointense volume in total baseline nonenhancing T2 lesions over 48 weeks of treatment.

Synthetic preparations for Compound 1 and suitable formulations for Compound 1 are described in U.S. Pat. No. 9,340,527, the entire teachings of which are incorporated herein by reference.

The two substituents on the cyclohexyl group in Compound 1 have a cis configuration relative to each other. When referring to Compound 1 by name or structure, its stereochemical purity is at least 90%, at least 95%, at least 98% or at least 99% by weight. Stereochemical purity is the weight ratio of compound in the cis configuration over the sum of the compound in the cis and trans configuration.

The invention is illustrated by the following examples, which is not intended to be limiting in any way.

### EXEMPLIFICATION

### Example 1—Compound 1 Inhibits DHCR7 Activity as Evidenced by the Accumulation of 7-DHC

Forty-two healthy volunteers received Compound 1 QD for 28 days (or before early withdrawal), with 6 individual participants per cohort receiving 1 mg (Cohort 1), 3 mg (Cohort 2), 10 mg (Cohort 3), 30 mg (Cohort 4), 60 mg loading dose with 10 mg maintenance dosing (Cohort 5) Compound 1, 60 mg (Cohort 6), or 90 mg loading dose on Day 1 and Day 2 with 30 mg maintenance dosing (Cohort 7). There were also 14 participants who received placebo in the study; 1 participant in Cohort 4 was misdosed on Day 19 and appeared to have received at least one 30 mg dose of Compound 1. The following protocol deviations were reported for participants in Cohorts 1 through 5: they received predose water ad lib (not restricted for 1 hour before and 1 hour after dose) and a meal administered 30 minutes postdose (not 4 hours postdose). A total of 49 participants completed treatment, including 37 participants who received active treatment.

Participants received the first dose of study treatment (Compound 1 or placebo) on Day 1 and continued to receive once-daily study treatment through Day 28. Participants remained in the clinic throughout the dosing period. Participants were discharged on Day 29 after completing all assessments.

Blood was periodically withdrawn from each volunteer and immediately stored at −80° C. For measuring metabolite concentrations in human serum, the samples were centrifuged and the resulting supernatant was used for further analysis.

Free oxysterols were extracted from the samples with methanol using the Biocrates Kit filter plate. The plate was loaded with an internal standard mixture beforehand. The metabolite concentrations were determined by UHPLC-MS/MS with multiple reaction monitoring (MRM) in a positive mode using a SCIEX API 5500 QTRAP® (AB SCIEX, Darmstadt, German) instrument with electrospray ionization (ESI). Data were quantitated using appropriate mass spectrometry software and imported in Biocrates Met/DQ™ software for further analysis.

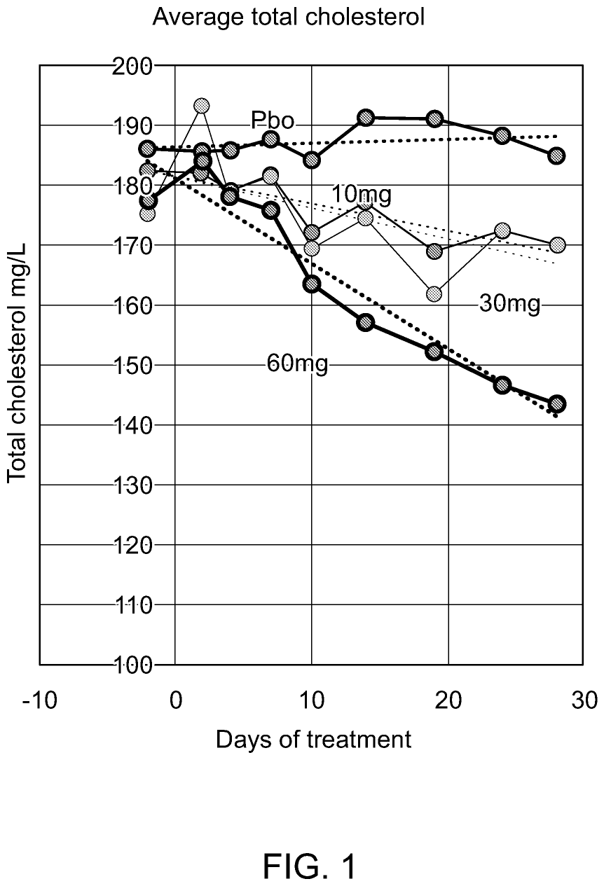

Circulating mean total cholesterol levels in the healthy volunteers are shown in FIG. 1, which demonstrates a gradual, time- and dose-dependent reduction in total circulating cholesterol.

Based on these observations, a population PK/PD model was developed using Monolix to describe circulating cholesterol concentration as a function of plasma concentration of Compound 1 and exposure. The model was developed from cholesterol data from the study described above (Study 1) as well as two additional clinical studies (Study 2 and Study 3) in healthy volunteers. In Study 2, thirty healthy volunteers received a single oral dose of Compound 1 with 6 individual participants per cohort receiving 3 mg (Cohort 1), 10 mg (Cohort 2), 30 mg (Cohort 3), 60 mg (Cohort 4), 100 mg (Cohort 5). There were also 9 participants who received placebo in the study. In Study 3, 8 healthy adult volunteers received a single dose of 30 mg of Compound 1.

The concentration of circulating cholesterol in this model is reduced at all daily doses higher than 10 mg. Although data variability impacts the predicted effects, the model shows clear evidence for a dose-dependent reduction in circulating cholesterol concentration. The EC₅₀ for this reduction is about 3 μg/mL, which approximates the steady-state concentration of Compound 1 at a daily dose of 60 mg.

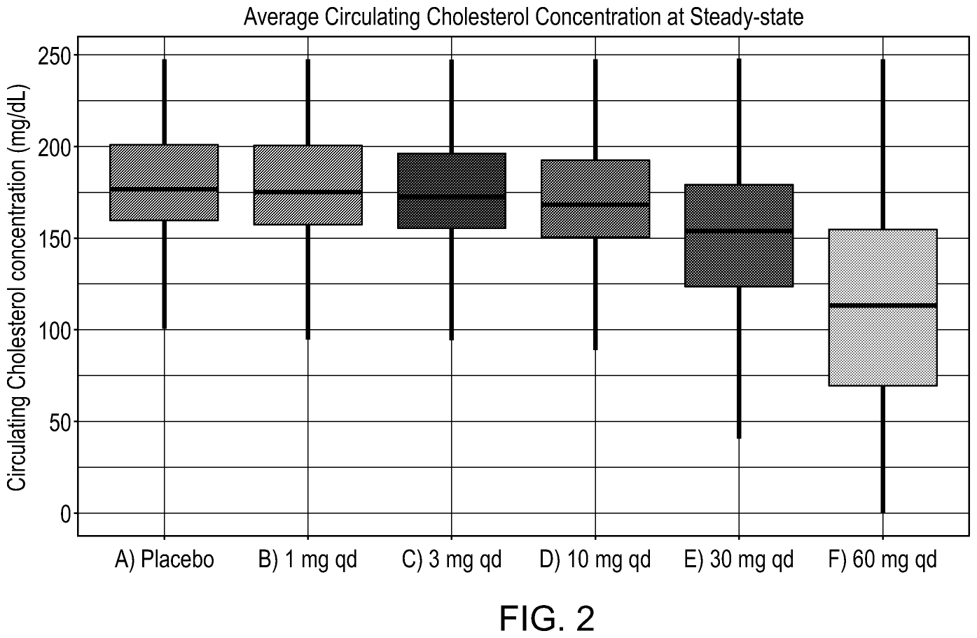

Healthy volunteers receiving the 60 mg dose in Study 1 had an average decrease of up to approximately 20% in total cholesterol, which is currently believed to affect the low-density lipoprotein fraction to a greater extent than the high-density lipoprotein fraction. The model predicts that circulating cholesterol could decrease by approximately 35% at a dose level of 60 mg QD. Higher doses of Compound 1 could result in a greater reduction of circulating cholesterol levels but are also likely to cause neutropenia. The reduction in circulating cholesterol is predicted to occur over a time frame commensurate with the increase to steady-state concentrations of Compound 1 in plasma (approximately 15 days), after which circulating cholesterol levels are predicted to stabilize for the duration of dosing. The model predicts that after discontinuation of Compound 1 treatment, circulating cholesterol will return to baseline levels within approximately 30 days. The model also predicts that both the rate of decrease and restoration of circulating cholesterol levels is limited by the accumulation and clearance rate of Compound 1 in plasma. The predicted steady-state concentrations of circulating cholesterol during treatment with Compound 1 in the dose range used in clinical studies are shown in FIG. 2.

### Example 2—Compound 1 Caused Accumulation of 7-DHC, Reduction of Desmosterol and No Change in Cholesterol in Rat OPCs

Enriched populations of oligodendrocytes from female Sprague Dawley postnatal day 2 (P2) rats were grown in culture. Briefly, the forebrain was dissected and placed in Hank's buffered salt solution (HBSS) (Life technologies). The tissue was cut into 1 mm fragments and incubated at 37° C. for 15 min in 0.01% trypsin and 10 μg/ml DNase. Dissociated cells were plated on poly-D-lysine (PDL)-coated T75 tissue culture flasks and grown at 37° C. for 10 days in Dulbecco's Modified Eagle Medium (DMEM) with 20% fetal calf serum (Life technologies). Oligodendrocyte precursors (A2B5+) were collected by shaking the flask overnight at 200 rpm at 37° C., resulting in a 95% pure population. Cultures were maintained in a defined growth medium (high glucose DMEM, 0.1% BSA, 50 ug/ml Apo-transferrin, 5 ug/ml insulin, 30 nM sodium selenite, 10 nM biotin and hydrocortisone) with 10 ng/ml fibroblast growth factor/platelet-derived growth factor (FGF/PDGF) (Peprotech) for 2-3 days. For assessing the ability of Compound 1 to promote the differentiation of rat A2B5+ progenitor cells into mature myelin basic protein positive (MBP+) myelinating oligodendrocytes, A2B5+ cells were plated into 10-cm PDL coated culture plates in FGF/PDGF free growth medium supplemented with 10 ng/ml CNTF and 15 nM T3 and were immediately treated for with Compound 1. The cell pellets were collected at 24 hours and 72 hours in culture and stored at −80° C. The cell pellets were subsequently shipped to Metabolon (Morrisville, NC, USA) and maintained at −80° C. during shipping and storage until processed. The cell pellet samples were extracted with methanol under vigorous shaking for 2 min (Glen Mills GenoGrinder 2000) to precipitate protein and dissociate small molecules bound to protein or trapped in the precipitated protein matrix, followed by centrifugation to recover chemically diverse metabolites. The resulting extract were next aliquot and analyzed on Metabolon's HD4 platform. Several types of quality control samples, including recovery standards added prior to extraction, technical replicates from a pool combined from each experimental sample, process and solvent blanks, and spiked a cocktail of QC standards, were applied during sample preparation and analysis for quality assessment and filtering failed samples.

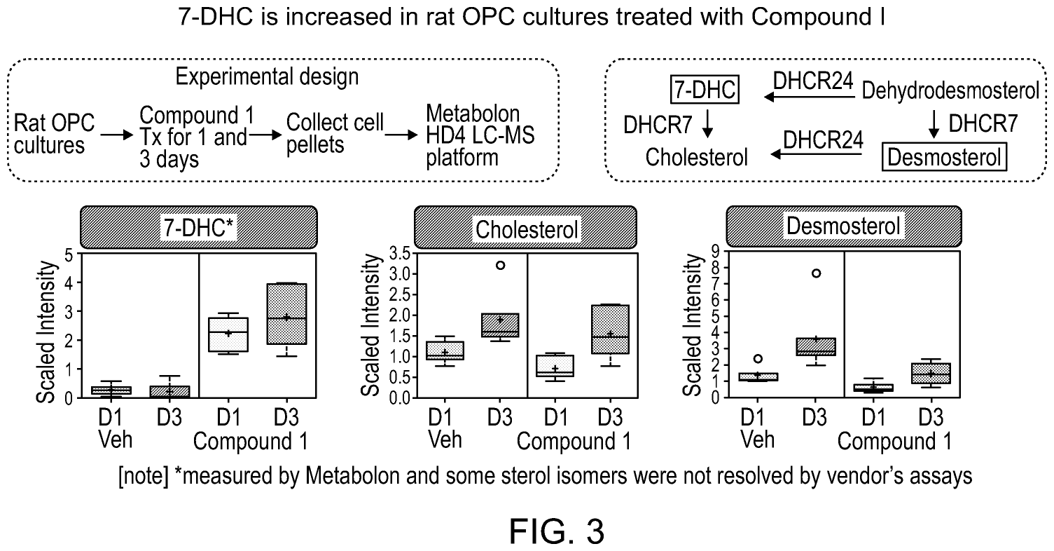

The Metabolon data revealed that Compound 1 treatment resulted in increased DHCR7 substrate 7-DHC accumulation and decreased or trendily decreased accumulation of DHCR7 products, desmosterol and cholesterol, in culture when comparing to the vehicle control of the same time point. At 24 hours, Compound 1 treated OPC culture showed 8.3 folds of 7-DHC (p=2.5e-5, q=0.005), 0.44 folds of desmosterol (p=0.002, q=0.15), and 0.64 folds of cholesterol (p=0.018, q=0.15). By 72 hours, Compound 1 treated OPC culture showed 12.86 folds of 7-DHC (p=8.1e-8, q=2.6e-5), 0.4 folds of desmosterol (p=0.001, q=0.022), and insignificant change of cholesterol. See FIG. 3.

The data suggest that although Compound 1 is shown to decrease peripheral cholesterol level in humans, it does not decrease cholesterol level in rat OPCs.

### Example 3—Compound 1 Enhanced Remyelination in the Lysophosphatidyl Choline Induced Spinal Cord and Corpus Callosum Demyelination and the Cuprizone Demyelination Animal Models

Lysophosphatidyl choline (hereinafter “LPC”) induced spinal cord and corpus callosum demyelination models are simple in vivo systems for investigating remyelination. LPC was injected into the dorsal column or corpus callosum of 9-week-old adult female SD rats on Day 0. Compound 1 was administered daily starting on Day 3 by oral dosing. Animals were sacrificed on Day 9, and the region of the spinal cord encompassing the lesion was excised and sectioned. Remyelinated axons were determined by quantifying the myelinated fibers from toluidine blue staining of 1-μm thin sections. The numbers of myelinated axons from 10 microscopic fields per animal and 3 animals per group were counted.

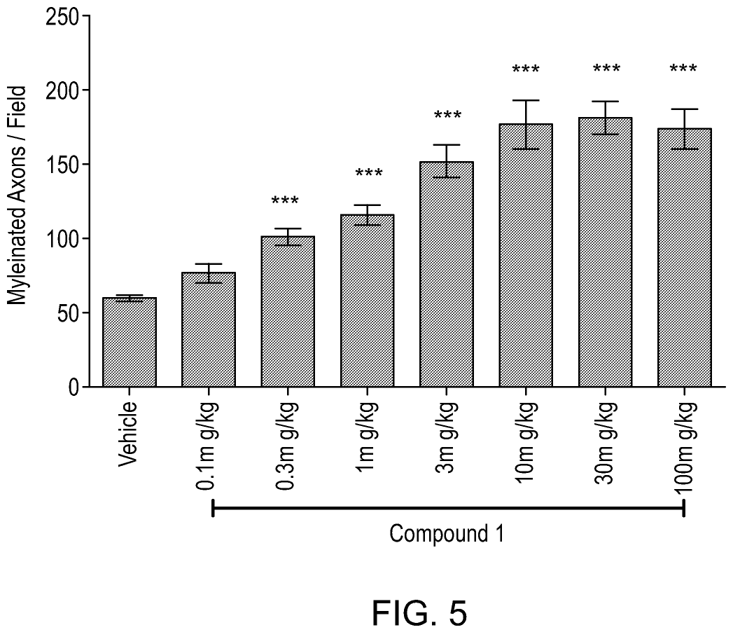

Tissue sections from control-treated animals showed large lesions with extensive areas of demyelination as evident from the absence of stained remyelinated axons in the lesion area. In contrast, Compound 1 enhanced remyelination in a dose dependent manner. The minimal efficacious dose was 0.3 mg/kg, and the dose for 90% of maximum observed biologic effect (ED₉₀) was 3 mg/kg, which was determined by counting the remyelinated axon fibers in the lesions. Remyelination was visualized by toluidine blue staining of the demyelinated spinal cord dorsal column. Results are shown in FIG. 5. *** p<0.001. Error bars denote the standard error of the mean.

Compound 1 induced remyelination in the corpus callosum after demyelination caused by cuprizone feeding. In this model, lesions are typically detectable in the corpus callosum of mice after 4 weeks of cuprizone feeding.

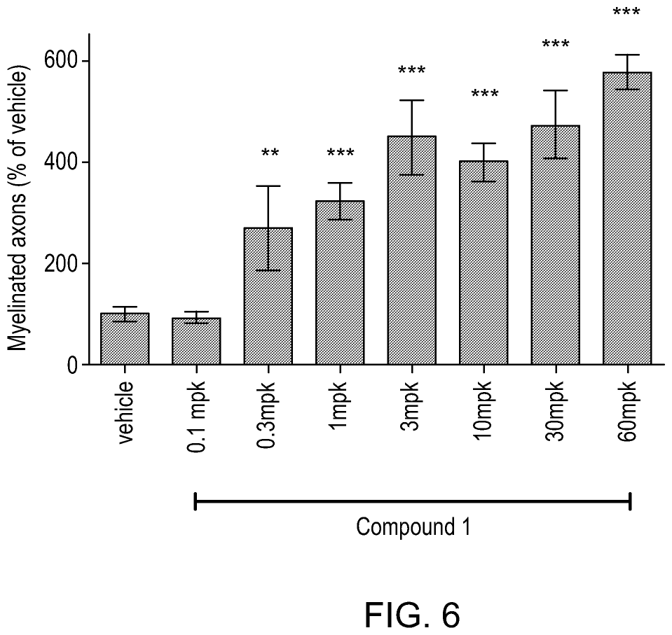

Nine week-old C57/BL6 mice were fed with chow pellets containing 0.3% cuprizone (Harlan) and injected with rapamycin intraperitoneally for 6 weeks (10 mg/kg, 5 days/week). Animals were treated with Compound 1 by oral gavage daily in the last 2 weeks of cuprizone/rapamycin treatment. Animals were sacrificed at the end of the last treatment and brains were dissected to determine the effect of Compound 1 on remyelination in the corpus callosum (white matter) and cerebral cortex regions. Coronal slices (septostriatal section, 1 mm thickness) through the corpus callosum were cut, then cut on the midsagittal plane and embedded in epon, oriented to visualize the entire cross-section of the midsagittal corpus callosum. Myelinated axons in the corpus callosum lesions were quantified by toluidine blue staining. ** p<0.01, *** p<0.001. The results are shown in FIG. 6. Compound 1 showed a dose dependent enhancement of remyelination. Error bars denote the standard error of the mean. N=39 for the study.

### Example 4—Compound 1 Enhanced Myelin Expression in a Rat Dorsal Root (DRG)/Oligodendrocytes Assay

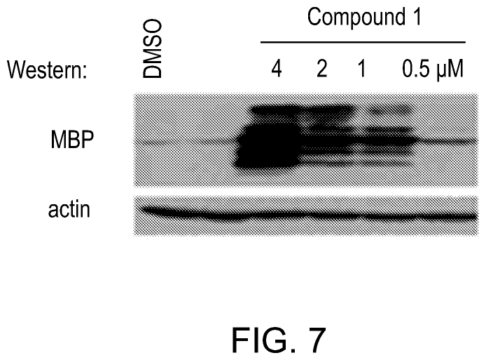

Embryonic dorsal root ganglia (DRG) dissected from embryonic day 14 (E14) to day 17 (E17) Sprague Dawley rats, were plated on poly-L-lysine (100 μg/ml)-coated cover slips for 2 weeks and grown in Neurobasal medium supplemented with B27 (Life technologies). To remove proliferating glial cells, cultures were pulsed twice with fluorodeoxyuridine (20 μM) from days 2-6 and from days 8-10. Then rat A2B5+ oligodendrocytes were added to DRG neuron drop cultures in the presence or absence of compounds at 37° C. with 5% CO2 for 13 days. The culture media (Neurobasal medium supplemented with B27 and 100 ng/ml nerve growth factor (NGF)) with fresh compounds were changed twice per week. Myelination is determined by Western blotting to quantify MBP level. Compound 1 promoted myelination in OPC/DRG co-culture assay in a dose-dependent manner (see FIG. 7).

### Example 5—Compound 1 Enhanced Human iPSC-derived Oligodendrocyte Progenitor Cell Differentiation and Myelination

Human induced pluripotent stem cell (iPSC) derived OPCs were maintained in proliferation media, composed of advanced DMEM-F12 supplemented with N2, B27, Glutamax, 5 mg/mL heparin (Sigma), 1 μM purmorphamine (Merck), 20 ng/ml FGF/PDGFa (Peprotech), 10 ng/ml IGF (Peprotech), and 60 ng/ml T3 (Sigma). To allow OPC differentiation, purmorphamine and FGF/PDGF were removed from the culture media. Compound 1 (0.02 and 0.2 μM) was incubated with human iPSC-derived OPCs in the differentiation media for 40 days.

Embryonic dorsal root ganglia (DRG), dissected from embryonic day 14 (E14) to day 17 (E17) Sprague Dawley rats, were plated on poly-L-lysine (100 μg/ml)-coated cover slips for 2 weeks and grown in Neurobasal medium supplemented with B27 (Life Technologies). To remove proliferating glial cells, cultures were pulsed twice with fluorodeoxyuridine (20 μM) from days 2-6 and from days 8 to 10. Then human OPCs, prepared as described above, were added to DRG neuron drop cultures. The culture media for rat OPC-DRG co-culture is Neurobasal medium supplemented with B27 and 100 ng/ml NGF with fresh compounds changed twice per week. The culture media for human OPC-rat DRG co-culture is the proliferation media for human OPCs without purmorphamine and FGF/PDGF. Compound 1 (0.02, 0.2, or 2 μM) or DMSO control treatment started 1 day after OPCs were added to DRG culture. To visualize myelination, cultures were fixed by 4% paraformaldehyde (PFA) and labeled with anti-myelin basic protein (MBP) antibodies to identify changes in myelination by ICC.

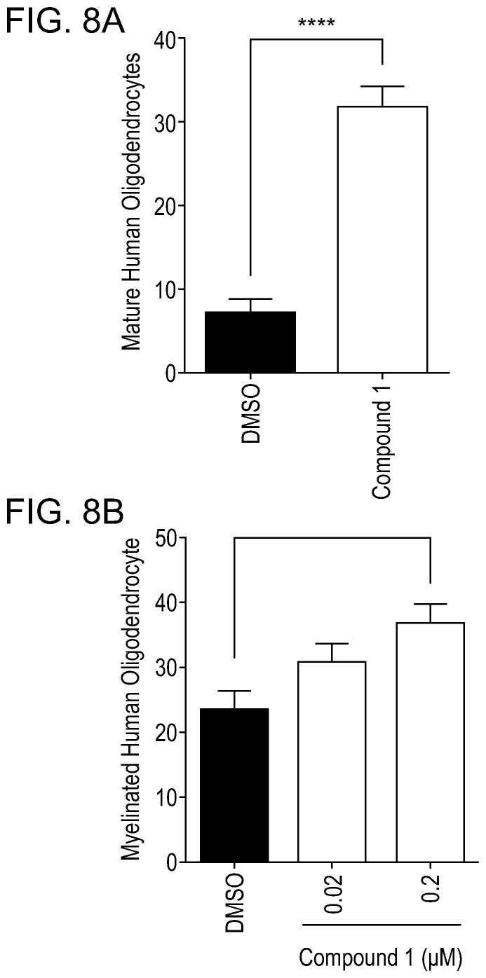

The results are shown in FIG. 8. As can be seen from FIG. 8, Compound 1 increased the number of MBP+ cells and the amount MBP+ myelinated axon clusters relative to control.

### Example 6—S1P4 Expression in Neural Cells Rat OPCs but not Human CNS Cells from MS Tissue

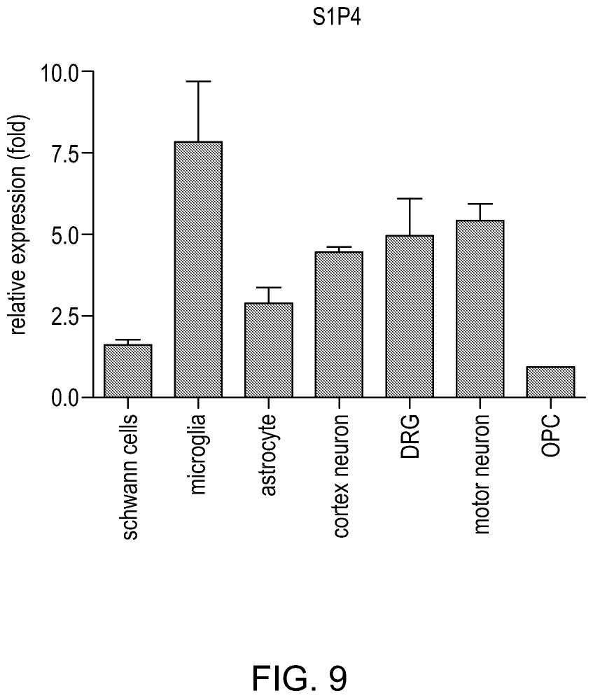

Expression of S1P4 in rat neural cells was evaluated by quantitative PCR analysis of first-strand complementary DNA synthesized from RNA obtained from rat neural cells. Rat actin messenger RNA (mRNA) was amplified as an internal control. The results are shown in FIG. 9, specifically that there is significant S1P4 messenger ribonucleic acid (mRNA) levels in rat CNS and peripheral nervous system cells, including in OPCs. The abundance of sphingosine-1-phosphate receptor 4 (S1P4) mRNA is presented relative to the normalized amount in A2B5+ oligodendrocyte progenitor cells (OPCs) [level as 1]. DRG=dorsal root ganglion.

Autopsy and neuropathology evaluation of donor subjects with an in life diagnosis of multiple sclerosis was performed according to standard protocols. Briefly, regions containing active or chronic active lesions, identified by gross examination of autopsy tissues, as well as normal appearing white matter, were dissected according to a tissue-banking grid system. Active and chronic active lesions were defined as demyelinated (using Anti-MOG immunohistochemistry (IHC) on select tissue blocks) regions of white or grey matter with either diffuse (active) or multifocal (chronic active) infiltrates of oil-red O positive lipid-laden macrophages/microglia. Tissues were fixed in 10% NBF for 3 weeks, immersed in a 30% sucrose medium for 2 weeks, then snap-frozen in isopentane cooled over dry ice before long-term storage at −80° C. Donors diagnosed with secondary progressive MS and with blocks of brain or spinal cord containing active, chronic active and normal appearing white matter (NAWM) were selected for evaluation of S1PR4 expression using in situ hybridization (ISH).

Fixed frozen issues were thawed, then submerged in 10% NBF and fixed overnight at room temperature prior to processing and embedding in paraffin. Blocks were sectioned at 5 um. ISH for PPIB (Cyclophilin B) was performed on NAWM blocks to determine whether adequate signal could be demonstrated. Slides were examined for PPIB signal. There was multifocal to diffuse signal, most robust in grey matter. In some specimens, large regions of PPIB signal were missing. These regions sometimes correlated with the presence of an active or chronic active, demyelinated region. Donors with NAWM blocks with adequate and relatively uniform PPIB signal were selected for S1PR4 ISH, as detailed in the table below.

| | |
|---|---|
| Total blocks examined | 21 |
| pMS blocks examined | 19 |
| non-neurologic control donor blocks examined | 2 |
| # MS donors | 7 |
| # non-neurologic control donors | 2 |

S1PR4 ISH was performed using the automated RNAscope assay on a Leica Biosystems' BOND RX platform. It was performed according to the manufacturer's instructions. ISH for S1PR4 mRNA expression and controls was performed with the following probes/reagents in the table below. Probes and ISH reagents used in the study were obtained from Advanced Cell Diagnostics, Inc (ACD). The tissue quality of the study samples was examined by positive control probe-PPM (Cyclophilin B), and the specificities of the probes were assessed by negative control probe-dapB (bacterial gene), respectively. S1PR4 reacted ISH slides were scanned using a Panoramic whole slide imager. Digital images were examined by a board certified veterinary pathologist for the presence of signal in various cells types and anatomic regions. For example, detection of S1PR4 ISH signal was described in meningeal infiltrates, vascular cuffs, and within CNS parenchyma.

| Probe/kit | Cat NO. |
|---|---|
| RNAscope ® 2.5 LS Probe Hs- S1PR4 | 475608 |
| RNAscope ® 2.5 LS Probe Hs-PPIB | 313908 |
| RNAscope ® 2.5 LSx Reagent Kit-RED | 322750 |
| RNAscope ® 2.5 LS Negative Control Probe_dapB | 312038 |

The following control experiments were run on a subset (n=6) of tissue blocks from MS donors described above. RNase treatment: The assay was performed according to ACD's instructions. Briefly, after RNAscope® Protease digestion, the tissues were treated with RNase (RNase-Qiagen catalog #19101) for 30 minutes at 40° C., and then were subjected to probe hybridization and the remaining RNA scope procedures. S1PR4 sense probe: custom designed by ACD scientists to complement precisely the S1PR4 sequence used for the antisense probe described above. S1PR4 gene is comprised of a single exon. This makes the antisense probe more likely to hybridize to genomic DNA.

| Probe | Cat NO. |
|---|---|
| RNAscope ® 2.5 LS Probe -Hs-S1PR4-sense | 833608 |
| RNAscope ® 2.5 LS Probe -Hs-PDFGRa | 604488 |

Image analysis of S1PR4 anti-sense, sense, RNase treated tissues, and PDGFRa on subset of tissue blocks (n=6): S1PR4 ISH signal detection in whole tissue and specific to nuclei and perinuclear area: ISH dots were detected with a customized algorithm in Visiopharm software using a combination of deep learning and conventional image analysis features. ISH signal specific to nuclei and perinuclear area were quantified by segmenting nuclear area as a separate ROI via hematoxylin counterstain.

Weak to moderate S1P4 signal was observed in perivascular cuffs and meninges, which was interpreted as deriving from infiltrating lymphocytes believed to be B lymphocytes. A positive-control staining with probe directed against the platelet-derived growth factor receptor indicated abundant expression in OPCs.

S1P4 receptor expression in humans is restricted to cells of hematopoietic origin with highest expression occurring in neutrophils and monocytes.

### Example 7—Effect of Compound 1 on Neutrophil Count

Two of 56 participants in the study described in Example 1 developed CTCAE grade 3 neutropenia (<1.0 to 0.5×109 neutrophils/L). Neutropenia was reported as an AE, which led to discontinuation of study drug, in these 2 participants:
1 participant in Cohort 6 (Compound 1 60 mg)
1 participant in Cohort 7 (Compound 1) 90 mg loading dose followed by 30 mg maintenance dose)

In addition to neutropenia in these 2 participants, a dose-dependent decline in absolute neutrophil count from baseline was observed in laboratory data from 22 other participants in the study who received doses ≥30 mg:
6 participants in Cohort 4 (Compound 1 30 mg)
6 participants in Cohort 5 (Compound 1 60 mg loading dose followed by 10 mg maintenance dose)
5 participants in Cohort 6
5 participants in Cohort 7

The extent of neutrophil count decline in this study at low Compound 1 doses (<30 mg, i.e., Cohorts 1, 2, and 3) was indistinguishable from placebo control, suggesting minimal Compound 1 effect on neutrophils at these concentrations. With the exception of the patient who discontinued treatment due to CTCAE grade 3 neutropenia, all samples in the 60 mg QD dose remained throughout the study in the normal range for absolute monocyte counts.

1. A method of treating a human subject with multiple sclerosis (MS), comprising administering to the subject from 10 mg to 60 mg per day of Compound 1: 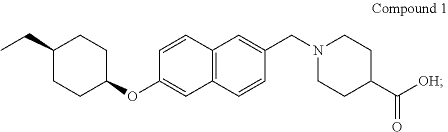 [SMILES: CC[C@H]1CC[C@@H](Oc2ccc3cc(CN4CCC(C(=O)O)CC4)ccc3c2)CC1]
or an amount of a pharmaceutically acceptable salt thereof equivalent to 10 mg to 60 mg per day of Compound 1.

2. The method of claim 1, wherein the subject is administered from 10 to 60 mg per day of Compound 1.

3. The method of claim 1, wherein the MS is in the relapsing remitting stage or the secondary progressive stage.

4. (canceled)

5. The method of claim 1, further comprising the step of i( administering to the human subject an effective amount of an additional agent effective in the treatment of MS or ii) administering to the human subject an effective amount of interferon-β1 or Glatiramer acetate.

6. (canceled)

7. The method of claims 2, comprising administering to the subject 10 mg per day of Compound 1, or 30 mg per day of Compound 1, or 60 mg per day of Compound 1.

8. (canceled)

9. (canceled)

10. A method of treating a human subject with multiple sclerosis (MS), comprising administering to the subject an effective amount of Compound 1: 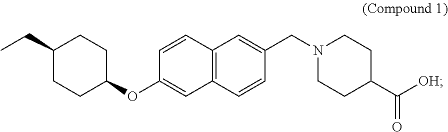 [SMILES: CC[C@H]1CC[C@@H](Oc2ccc3cc(CN4CCC(C(=O)O)CC4)ccc3c2)CC1]
or a pharmaceutically acceptable salt thereof, in the absence of a cholesterol lowering drug.

11. The method of claim 10, wherein the subject is being treated with a cholesterol lowering drug, and treatment with the cholesterol lowering drug is terminated prior to initiation of treatment with Compound 1 or a pharmaceutically acceptable salt thereof.

12. The method of claim 10, wherein treatment with the cholesterol lowering drug is terminated at least 6 days prior to initiation of treatment with Compound 1 or a pharmaceutically acceptable salt thereof.

13. The method of claim 10, wherein the cholesterol lowering drug is a statin, a PCSK9 inhibitor, a selective cholesterol absorption inhibitor, a bile acid sequestrant, a fibrate or a lipid-lowering therapy.

14. The method of claim 10, wherein the subject is administered from 10 mg to 60 mg per day of Compound 1 or an amount of a pharmaceutically acceptable salt thereof that is equivalent to from 10 mg to 60 mg per day of Compound 1.

15. (canceled)

16. The method of claim 10, wherein the subject is administered 10 mg/day of Compound 1, or 30 mg/day of Compound 1, or 60 mg/day of Compound 1.

17. (canceled)

18. (canceled)

19. The method of claim 10, wherein the MS is in the relapsing remitting stage or in the secondary progressive stage.

20. (canceled)

21. The method of claim 10, further comprising the step of i) administering to the human subject an effective amount of an additional agent effective in the treatment of MS or ii) administering to the human subject an effective amount of interferon-⊕1 or Glatiramer acetate.

22. (canceled)

23. A method of treating a human subject with multiple sclerosis (MS), wherein the subject is being treated with an effective amount of a cholesterol lowering drug, comprising the steps of:
i) administering to the subject an effective amount of Compound 1: 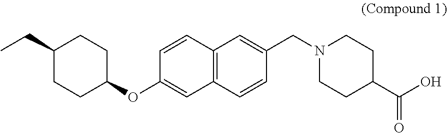 [SMILES: CC[C@H]1CC[C@@H](Oc2ccc3cc(CN4CCC(C(=O)O)CC4)ccc3c2)CC1]
or a pharmaceutically acceptable salt thereof;
ii) assessing the subject's plasma cholesterol level;
iii) if the subject's plasma cholesterol level is outside of a target range, adjusting the amount of cholesterol lowering drug administered to the subject to bring the subject's plasma cholesterol level within the target range.

24. The method of claim 23, wherein steps ii) and iii) are repeated until the subject's plasma cholesterol level is within the target range, wherein the target range is between 100 mg/dL and 200 mg/dL or between 125 mg/dL and 200 mg/dL.

25. (canceled)

26. (canceled)

27. The method of claim 23, wherein the cholesterol lowering drug is a statin, a PCSK9 inhibitor, a selective cholesterol absorption inhibitor, a bile acid sequestrant, a fibrate or a lipid-lowering therapy.

28. The method of claim 23, wherein the subject is administered from 10 mg to 60 mg per day of Compound 1 or an amount of a pharmaceutically acceptable salt thereof that is equivalent to from 10 mg to 60 mg per day of Compound 1.

29. (canceled)

30. The method of claim 23, wherein the subject is administered 10 mg/day of Compound 1, or 30 mg.day of Compound 1, or 60 mg/day of Compound 1.

31. (canceled)

32. (canceled)

33. The method of claim 23, wherein the MS is in the relapsing remitting stage.

34. (canceled)

35. The method of claim 23, further comprising the step of i) administering to the human subject an effective amount of an additional agent effective in the treatment of MS or ii) administering to the human subject an effective amount of interferon-⊕1 or Glatiramer acetate.

36. (canceled)
